(19) United States
(12) Patent Application Publication
(10) Pub. No.: US 2015/0011450 A1
(43) Pub. Date: Jan. 8, 2015

(54) Amphoteric Ter-Polymers For Use in Personal Care Compositions

(71) Applicant: The Procter & Gamble Company, Cincinnati, OH (US)

(72) Inventors: John David CARTER, Mason, OH (US); Dipesh Mukesh PATEL, Cincinnati, OH (US)

(21) Appl. No.: 14/322,573

(22) Filed: Jul. 2, 2014

Related U.S. Application Data
(60) Provisional application No. 61/842,744, filed on Jul. 3, 2013.

(51) Int. Cl. A61K 8/72; A61Q 5/12; A61Q 5/02

(52) U.S. Cl. CPC A61K 8/72; A61Q 5/02; A61Q 5/12; A61K 2800/59
USPC 510/122; 510/119

(57) ABSTRACT

The present invention is directed to a personal care composition comprising: from about 5% to about 50% of a surfactant; from about 0.01% to about 10% of a conditioning agent; from about 0.05 to about 5 weight percent of the personal care composition of an amphoteric ter-polymer of the following:
i.) a cationic monomer encompassed by formula (I),

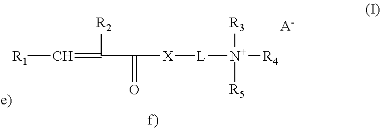

in which:
R₁ and R₂ are independently hydrogen or methyl, R₃, R₄ and R₅ are independently linear or branched C₁-C₄ alkyl radicals, X is NH, NR₆ or oxygen, wherein R₆ is C₁-C₄ alkyl, L is CₙH₂ₙ, n is an integer from 1 to 5, and A⁻ is an anion derived from an organic or inorganic acid, such as a methosulphate anion or halide, such as chloride or bromide,
ii.) at least one anionic monomer selected from the group consisting of ethylenically unsaturated carboxylic acid and sulfonic acid containing monomers; and
iii.) a diallylamine monomer defined by formula (II),

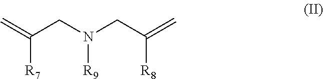

in which:
R₇ and R₈ are independently hydrogen or C₁-C₄ alkyl, and R₉ is hydrogen, branched or linear C₁-C₃₀ alkyl, *-[AO]ₘ—R₁₀, C₁-C₃₀ alkoxy, hydroxy substituted alkyl, alkylphenyl, carboxyalkyl, alkoxyalkyl and carboxyamidalkyl; AO is a C₁-C₄ alkylene oxide or mixtures of two or more types thereof, it being possible for the two or more types to be attached to one another in block form or in random form, m is an integer from 2 to 200, R₁₀ is hydrogen or methyl; and wherein the ratio of cationic monomer i) to anionic monomer ii) is from about 5 to about 1.

### FIELD OF THE INVENTION

The present invention relates to a personal care composition containing an amphoteric ter-polymer, an anionic surfactant, an aqueous carrier, wherein the composition provides wet and dry conditioning benefits, deposits conditioning agents uniformly across hair type and do not result in multiple wash cycle performance tradeoffs.

### BACKGROUND OF THE INVENTION

Conditioning shampoos or “2 in 1” hair products comprising a detersive surfactant and hair conditioning agents are known. These personal care compositions typically comprise an anionic detersive surfactant in combination with a conditioning agent such as a silicone, hydrocarbon oil, fatty esters etc. These products have become more popular among consumers as a means of conveniently obtaining hair conditioning and cleansing performance from a single product.

Many conditioning personal care compositions, however, do not provide sufficient deposition of conditioning agents onto hair or skin during the application process and if deposition is possible, it is only possible in formulations with relatively low levels of anionic surfactant. Without adequate deposition, large proportions of conditioning agent are rinsed away during the application process and therefore provide little or no conditioning benefit. Without sufficient deposition of the conditioning agent on the hair or skin, relatively high levels of conditioning agents may be needed. Such high levels of a conditioning agent, however, can increase raw material costs, reduce lathering, and present product stability concerns. Additionally, limitations on total anionic surfactant in order to form coacervate can limit the lather potential of a composition, or result in the need for higher levels of less cost effective amphoteric surfactants in order to achieve good lather.

One known method for improving deposition of a hair conditioning agent onto hair involves the use of specific cationic deposition polymers. These polymers may be synthetic, such as those based on cationic acrylamide architectures, but are most commonly natural cellulosic or guar polymers that have been modified with cationic substituents.

The intrinsic level of conditioning provided by conventional cationic polymers such as those derived from cellulosics, guars and synthetic acrylamides is relatively low, especially in the wet state. Additionally, the high cationic polymer levels required to provide in-use benefits may result in consumer noticeable performance tradeoffs. Moreover, when used as a deposition aid, these performance tradeoffs may be exacerbated after multiple wash cycles due to over deposition of the hair conditioning agent on more hydrophobic substrates (e.g. root vs. tip, virgin brown vs. bleach damaged).

Net, there is a need for polymer systems that provide both effective wet and dry conditioning by themselves, act as efficient deposition aids of hair care benefits agents across hair type and do not result in multi-cycle tradeoffs such as buildup, weighdown, and lack of clean feel.

Surprisingly, ter-polymers of the present invention provide robust wet and dry conditioning benefits when formulated alone, deposit conditioning agents efficiently and uniformly across hair type and do not result in build-up or feel negatives over multiple wash cycles. When combined with other cationic polymers, terpolymers of the present invention have been found to further improve the in-use experience without any multi-cycle trade-offs.

### SUMMARY OF THE INVENTION

According to an embodiment of the present invention, it is directed to a personal care composition comprising: from about 5% to about 50% of a surfactant; from about 0.01% to about 10% of a conditioning agent; from about 0.05 to about 5 weight percent of the personal care composition of an amphoteric ter-polymer of the following:
i.) a cationic monomer encompassed by formula (I),

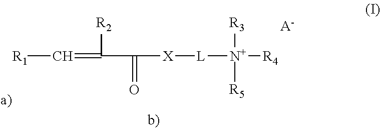

in which:
R₁ and R₂ are independently hydrogen or methyl, R₃, R₄ and R₅ are independently linear or branched C₁-C₄ alkyl radicals, X is NH, NR₆ or oxygen, wherein R₆ is C₁-C₄ alkyl, L is CₙH₂ₙ, n is an integer from 1 to 5, and A⁻ is an anion derived from an organic or inorganic acid, such as a methosulphate anion or halide, such as chloride or bromide,
ii.) at least one anionic monomer selected from the group consisting of ethylenically unsaturated carboxylic acid and sulfonic acid containing monomers; and
iii.) a diallylamine monomer defined by formula (II),

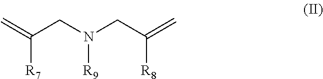

in which:
R₇ and R₈ are independently hydrogen or C₁-C₄ alkyl, and R₉ is hydrogen, branched or linear C₁-C₃₀ alkyl, *-[AO]ₘ—R₁₀, C₁-C₃₀ alkoxy, hydroxy substituted alkyl, alkylphenyl, carboxyalkyl, alkoxyalkyl and carboxyamidalkyl; AO is a C₁-C₄ alkylene oxide or mixtures of two or more types thereof, it being possible for the two or more types to be attached to one another in block form or in random form, m is an integer from 2 to 200, R₁₀ is hydrogen or methyl; and wherein the ratio of cationic monomer i) to anionic monomer ii) is from about 5 to about 1.

### DETAILED DESCRIPTION OF THE INVENTION

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography “QS” means sufficient quantity for 100%.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

“Kit,” as used herein, means a packaging unit comprising a plurality of components. An example of a kit is, for example, a first composition and a separately packaged second composition. Another kit may comprise a first composition and an energy delivery device. A different kit may comprise three different types of separately packaged composition and a hair styling implement. A further kit may comprise application instructions comprising a method and a composition/formulation.

The term “coacervate” as used herein, means the complex which forms between surfactant and polymer that may either be soluble or insoluble in the neat composition, typically forming an insoluble complex in the neat composition, and which may become less soluble upon dilution and thus yielding an increase in its level of phase separation or precipitate in solution.

The term “charge density” as used herein, means the ratio of the number of positive charges on a monomeric unit (of which a polymer is comprised) to the M.Wt. of said monomeric unit. The charge density multiplied by the polymer M.Wt. determines the number of positively charged sites on a given polymer chain. For cationic guars, charge density is measured using standard elemental analysis of percentage nitrogen known to one skilled in the art. This value of percentage nitrogen, corrected for total protein analysis, can then be used to calculate the number or equivalence of positive charges per gram of polymer. For the cationic copolymers, the charge density is a function of the monomers used in the synthesis. Standard NMR techniques know to one skilled in the art would be used to confirm that ratio of cationic and non-ionic monomers in the polymer. This would then be used to calculate the number or equivalence of positive charger per gram of polymer. Once these values are know, the charge density is reported in milliequivalence (meq) per gram of cationic polymer.

The term “(meth)acrylamide” as used herein means methylacrylamide or acrylamide. The term “(meth)acrylic acid” as used herein means acrylic acid or methacrylic acid.

Liquid crystals are also known as anisotropic fluids, a fourth state of matter, polymer association structure or mesophases. Those terms are used interchangeably. Lyotropic means a material is formed through changes in solution behavior (and hence by definition contains a solvent, for example water) of the ingredients. The changes involve thermal and solvation energies. The term “lyotropic liquid crystal” as used herein, refers to a liquid crystalline phase distinctive by the presence of birefringence (a non-limiting example of which is formation of maltose crosses) under polarized light microscopy. These are most easily observed in the absence of particles as some particles also demonstrate birefringence. In addition, the term “polymer liquid crystals”, as used herein, means “polymeric lyotropic liquid crystals” unless otherwise specified.

The ter-polymers complex with anionic surfactant at typical shampoo use levels and may form well dispersed microscopically phase separated particles or aggregates. These coacervates may exhibit birefringence under polarized light microscopy, behavior typical of an ordered mesophase or lyotropic liquid crystal. Without being bound by theory, the acrylic acid and diallylamine components are hypothesized to modulate the phase behavior and rheological profile of the polymer-surfactant complex and help elicit a softer/more lubricious sensory response. Moreover, theoretically, the amphoteric character facilitates more effective removal of these complexes in subsequent shampoo wash cycles as compared to typical high charge density systems.

In an embodiment of the present invention, liquid crystals have a particle size in the range of about 0.1 to about 50 micrometers, in an embodiment, in the range of about 0.5 to about 20 micrometers, and in another embodiment, in the range of about 1 to about 10 micrometers.

Polymers of the invention have surprisingly been found to be very effective deposition aide for conditioning materials such as silicones or other organic materials and other oils. Surprisingly, such polymers provide a more uniform root to tip deposition and performance across hair types such as virgin or damaged hair vs. typical isotropic and lyotropic cationic deposition aides.

Without being bound by theory, the more balanced deposition may be explained by the diversity of molecular interactions between such polymer-surfactant complexes and hair surfaces. In other words, the variety of functional groups present in the polymer chain contribute to their effective deposition both on virgin (hydrophobic)) and damaged (hydrophilic) surfaces.

In an embodiment of the present invention, for high charge density materials, the molecular weight of the ter-polymer may be in a range of about 100,000 to about 1,500,000; in a further embodiment, in a range of about 100,000 to about 1,000,000; in a further embodiment, in a range of about 200,000 to about 800,000; and in yet a further embodiment, in a range of about 250,000 to about 600,000.

In an embodiment of the disclosed ter-polymer systems is their use in low pH shampoo compositions, in one embodiment in the range 3.5-5.5, in a further embodiment is range of about 3.5 to about 4.5, in yet a further embodiment in the range 4.0-4.5. Enhanced conditioning benefits are observed versus typical shampoo compositions formulated in the pH range 5.5-7.5 as measured by subjective wet and dry combing and softness. Moreover, the reduced pH enhances the absolute deposition of silicone benefit agents without compromising volume and dry conditioning benefits such as clean hair feel, combability, shine and repair benefits.

Consistent with the amphoteric nature of the ter-polymers, it is believed the higher charge density at reduced pH results in a stronger polymer-surfactant interaction and more favorable in-use experience. Upon rinsing, the pH of the medium can increase by up to 3 units, weakening the ter-polymer-surfactant complex interaction with the hair surface such that the net deposition is maintained.

Personal cleansing or personal care composition comprising the conditioning ter-polymer along with a conditioning agent in one embodiment of the invention may exist in a complex coacervate form upon dilution of water or upon addition of the inventive ter-polymer to the formulation. The coacervate may include complexation with the conditioning agents such as a fatty amine, fatty amine oxide, fatty quaternary defined below, silicone, oil, or emollient also defined below.

### The Conditioning Ter-Polymer

The novel conditioning polymer of the invention is formed from at least three monomers,
i.) a cationic monomer encompassed by formula (I),

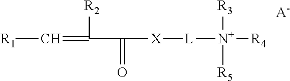

in which:
R₁ and R₂ are independently hydrogen or methyl, R₃, R₄ and R₅ are independently linear or branched C₁-C₄ alkyl radicals, X is NH, NR₆ or oxygen, and in an embodiment, wherein R₆ is C₁-C₄ alkyl, L is CₙH₂ₙ, n is an integer from 1 to 5, and A⁻ is an anion derived from an organic or inorganic acid, such as a methosulphate anion or halide, such as chloride or bromide,
ii.) at least one anionic monomer selected from the group consisting of ethylenically unsaturated carboxylic acid and sulfonic acid containing monomers; and
iii.) a diallylamine monomer defined by formula (II),

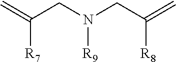

in which:
R₇ and R₈ are independently hydrogen or C₁-C₄ alkyl, and R₉ is hydrogen, branched or linear C₁-C₃₀ alkyl, *-[AO]ₘ—R₁₀, C₁-C₃₀ alkoxy, hydroxy substituted alkyl, alkylphenyl, carboxyalkyl, alkoxyalkyl and carboxyamidalkyl; AO is a C₁-C₄ alkylene oxide or mixtures of two or more types thereof, it being possible for the two or more types to be attached to one another in block form or in random form, m is an integer from 2 to 200, R₁₀ is hydrogen or methyl; and
iv.) optionally a crosslinking monomer,
wherein the formed ter-polymer is optionally at least partially neutralized or complexed with a fatty amine, fatty amine oxide or fatty quaternary.

The cationic monomer of formula (I) used in the inventive conditioning ter-polymer is for example selected from the group consisting of (meth)acryloyloxyethyl-N,N,N-trimethylammonium chloride, (meth)acryloyloxyethyl-N-ethyl-N,N-dimethylammonium monoethyl sulfate, (meth)acryloyloxyethyl-N,N,N-triethylammonium monoethyl sulfate, (meth)acryloylaminopropyl-N,N,N-trimethylammonium chloride, (meth)acryloylaminopropyl-N-ethyl-N,N-dimethylammonium monomethyl sulfate, (meth)acryloylaminopropyl-N,N-diethyl-N-methylammonium chloride, (meth)acryloylaminopropyl-N,N-diethyl-N-methylammonium monomethyl sulfate and mixtures thereof,

in an embodiment (meth)acryloylaminopropyl-N,N,N-trimethylammonium chloride, (meth)acryloylaminopropyl-N-ethyl-N,N-dimethylammonium monomethyl sulfate, (meth)acryloylaminopropyl-N,N-diethyl-N-methylammonium chloride, (meth)acryloylaminopropyl-N,N-diethyl-N-methylammonium monomethyl sulfate and mixtures thereof and
especially acryloylaminopropyl-N,N,N-trimethylammonium chloride, in an embodiment, X is NH.

The cationic monomer of formula (I) or component i) will for example make up at least about 10 to about 98 weight percent of the formed conditioning ter-polymer. Alternatively, for example the cationic monomer of formula (I) makes up about 40 to about 96 or about 40 to about 90 weight percent of the total weight of the formed ter-polymer. A minimum of about 40 or 50 weight % component i) is most typical.

The anionic monomers of component ii.) will typically contain carboxylic acids or sulfonic acid groups. For example, acrylic acid (AA), methacrylic acid (MAA), crotonic acid, 2-methyl crotonic acid, maleic acid, maleic anhydride, itaconic acid, itaconic anhydride, 2-acrylamido-2-methylpropane sulfonic acid (AMPSA), 2-methacrylamido-2-methylpropane sulfonic acid (MAMPSA)—and mixtures thereof are considered.

The anionic monomer of component ii.) are especially compounds of formula (III) or the anhydrides thereof:

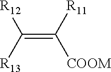

where
R₁₁ and R₁₂ are independently hydrogen or methyl, R₁₃ is hydrogen, methyl or a COOM group and M is hydrogen, a monovalent or divalent metal ion, ammonium or an organic ammonium ion.

The anionic monomer or component ii.) will, in an embodiment of the present invention, make up at least 2 to about 25, about 4 to about 20 weight percent, of the total weight of the formed conditioning polymer.

The molar ratio of components i.) and ii.) may vary from 1.5-12.0, in an embodiment 1.5-5.0, in a further embodiment 1.5-3.0. Thus the ter-polymer will always carry a cationic charge regardless of the pH of the medium in which the ter-polymer is dispersed or dissolved.

Component iii.) monomer is for example diallylamine (R₇, R₈, R₉=hydrogen), diallylmethylamine, or a monomer of formula IIa:

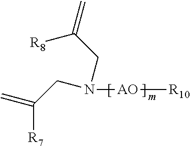

wherein AO is C₂-C₄ alkylene oxide such as ethylene oxide, propylene oxide, 1-butylene oxide, isomers of butylene oxide and mixtures thereof, it being possible for the two or more types of alkylene oxides to be attached to one another in block or in random form, R₇ and R₈ are as defined above, m is an integer from 1-200, in an embodiment 1-100, in a further embodiment 1-50 and R₁₀ is hydrogen or methyl. In an embodiment, alkylene oxides of monomer (IIa) are ethylene oxide and propylene oxide and mixtures thereof.

Such substituted alkyoxalated diallyamines are disclosed in U.S. Pat. Nos. 7,579,421 and 5,478,883 herein (VI) incorporated entirely by reference.

Diallylamines do not function as crosslinking agents although the monomers are diolefinic. Instead the monomer polymerizes to form a pyrrolidine ring as part of the polymer backbone as below.

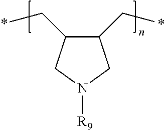

The component iii.) may make up from about 0.1 to about 40, about 0.5 to about 30, or about 1 to about 20 weight percent of the total weight of formed polymer.

The formed ter-polymer will for example carry a net positive charge. This net positive charge is primarily due to the monomer unit of formula (I) and is independent of the ter-polymer matrix or formulation environment. However, the diallylamine monomer component may also contribute to the total cationic charge of the formed ter-polymer when in an acidic environment. As shampoo formulations are typically slightly acidic, i.e. from about 5.5 to about 7.0, the diallylamine monomer unit of formula (II) will likely be protonated giving additional cationic charge to the formed ter-polymer. In more acidic formulations, i.e. from about 3.5 to about 5.5, the net positive charge of the formed ter-polymer will further increase due to protonation of the anionic monomer of formula (III).

The total charge density of the amphoteric ter-polymer will thus be dependent on the pH of the medium. Typically the charge density of the ter-polymer at a pH of 7.0 will vary from about 1.0 to about 3.0 at a pH of 7.0 and from about 2.0 to about 4.0 at a pH of 4.0.

The negative charge (from the anionic monomer or component ii.)) in the formed polymer may optionally be neutralized or form a complex or coacervate with a fatty amine, a fatty amine oxide, or a fatty quaternary either by adding the fatty amines, oxides or fatty quaternary during the polymerization process or after the polymerization process. For example, the negative charge produced by the acidic monomer may be neutralized prior to polymerization then polymerized. Alternatively, the fatty amine, or fatty amine oxide, or fatty quaternary may simply be added after formation of the amphoteric polymer. In an embodiment, the fatty amine, or fatty amine oxide, or fatty quaternary is added after the amphoteric polymer is formed if added at all.

The average molecular weight (M,) of the amphoteric conditioning ter-polymer or mixtures thereof ranges for example from about 100 to about 1,500 KDaltons. In an embodiment, the ter-polymer molecular weight falls in the range 200-1000 KDaltons and in a further embodiment 250-600 KDaltons.

In an embodiment of the present invention, the amphoteric ter-polymer may be used at about 0.05 to about 5 weight percent of the total personal care composition, in an embodiment at about 0.01 to about 3 weight percent of the total personal care composition, in another embodiment at about 0.1 to about 0.75 weight percent of the total personal care composition, and a further embodiment at about 0.1 to about 0.5 weight percent of the total personal care composition.

The amphoteric polymer may be either water soluble, water-swellable or water dispersible. The conditioning amphoteric polymer may optionally be cross-linked. Examples of crosslinkers are methylenebisacrylamide (MBA) and methylenebismethacrylamide. In an embodiment, a crosslinker may be added at about 300 ppm.

### Preparation of the Ter-polymer

The amphoteric conditioning polymers can be prepared in the conventional manner, e.g., by mass or solution polymerization. The polymerization may take place in an aqueous, solvent or aqueous-solvent mixed environment but in an embodiment, the reaction is to be carried out in a substantially aqueous environment. Possible solvents are DMSO, THF, DMF, ethyl, propyl, butyl, acetate, benzene, toluene, xylene, N-butanol, isobutanol, isopropanol, MEK, MIBK, acetone, etc. In an embodiment of the present invention, the polymerization is to be carried out in the absence of oxygen. In an embodiment, the monomers are polymerized using a radical reaction, by addition of peroxides, optionally in the presence of redox systems. Initiators such as ammonium persulfate are ideal as this initiator is highly water soluble. The polymerization time of the conditioning polymer depends on the temperature and the desired final product properties but is, in an embodiment, within the range of from 0.5 to 10 hours at temperatures ranging from about 50° C. to about 190° C. The polymerization can be carried out continuously, discontinuously or semicontinuously. In an embodiment of the present invention, a polymer chain can be obtained having random distribution of monomers, and in an embodiment, all of the monomers together will be added to the reaction mixture. This may be done in one portion or metered over time to control the rate of the reaction. On the basis of the reactivity of the monomers, which is known, a skilled artisan can control the polymerization so as to obtain the desired distribution. Non-limiting example of such preparation is found in US 2010/0226868 published on Sep. 9, 2010.

In an embodiment of the present invention, there may be various methods of formulating the amphoteric polymers in surfactant containing compositions. In one embodiment, may use as pre-diluted aqueous solution, added either before or after a surfactant system. In another embodiment, may pre-complex the amphoteric polymer(s) with an anionic surfactant and appropriate “stability enhancers” and the resulting dispersion added directly to a bulk surfactant.

In an embodiment of the present invention, the amphoteric ter-polymers are composed of at least three monomers,

i.) a cationic monomer of the formula:

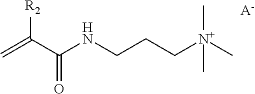

in which R₂ is hydrogen or methyl and A is an anion derived from organic or inorganic acid.
ii.) anionic monomers from the group of ethylenically unsaturated carboxylic acids of formula:

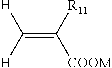

where R₁₁ is hydrogen or methyl and M is hydrogen, a monovalent metal ion, ammonium or organic ammonium ion.
iii.) one or more non-ionic monomers selected from diallylamine (DAA) or diallylamine derivatives defined by the formula:

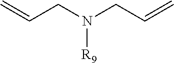

where R₉ is -[AO]ₘ—R₁₁, AO is ethyleneoxide or propyleneoxide or mixtures thereof, m=1-100, in an embodiment 1-50, in a further embodiment 1-20 and R₁₁ is hydrogen or methyl.
wherein the molar ratio of cationic monomer A to anionic monomer B falls in the range 1.5-12.0, in an embodiment 1.5-5.0, in a further embodiment 1.5-3.0
The ter-polymers are optionally at least partially neutralized with a fatty amine, fatty amine oxide, or fatty quaternary. In one embodiment, the ter-polymer weight falls in range 100-1500 KDaltons, in a further embodiment 200-1000 KDaltons, and in a further embodiment 250-600 KDaltons.

Deposition Polymer

In an embodiment, shampoo compositions of the present invention may additionally comprise one or more further cationic deposition polymers. These cationic deposition polymers can include at least one of (a) a cationic guar polymer, (b) a cationic non-guar galactomannan polymer, (c) a cationic tapioca polymer, (d) a cationic copolymer of acrylamide monomers and cationic monomers, and/or (e) a synthetic, non-crosslinked, cationic polymer, which may or may not form lyotropic liquid crystals upon combination with the detersive surfactant, (f) a cationic cellulose polymer. Additionally, the cationic deposition polymer can be a mixture of deposition polymers.

(1) Cationic Guar Polymers

According to an embodiment of the present invention, the shampoo composition comprises a cationic guar polymer, which is a cationically substituted galactomannan (guar) gum derivatives.

Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide the requisite cationic charge density described above.

According to one embodiment, the cationic guar polymer has a weight average M.Wt. of less than about 2.5 million g/mol, and has a charge density of from about 0.05 meq/g to about 2.5 meq/g. In an embodiment, the cationic guar polymer has a weight average M.Wt. of less than 1.5 million g/mol, or from about 150 thousand to about 1.5 million g/mol, or from about 200 thousand to about 1.5 million g/mol, or from about 300 thousand to about 1.5 million g/mol, or from about 700,000 thousand to about 1.5 million g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.7 meq/g.

According to one embodiment, the cationic guar polymer has a weight average M.Wt. of less than about 1 million g/mol, and has a charge density of from about 0.1 meq/g to about 2.5 meq/g. In an embodiment, the cationic guar polymer has a weight average M.Wt. of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol. from about 150 thousand to about 800 thousand g/mol, or from about 200 thousand to about 700 thousand g/mol, or from about 300 thousand to about 700 thousand g/mol, or from about 400 thousand to about 600 thousand g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.5 meq/g.

In an embodiment, the composition comprises from about 0.01% to less than about 0.7%, or from about 0.04% to about 0.55%, or from about 0.08% to about 0.5%, or from about 0.16% to about 0.5%, or from about 0.2% to about 0.5%, or from about 0.3% to about 0.5%, or from about 0.4% to about 0.5%, of cationic guar polymer (a), by total weight of the composition.

The cationic guar polymer may be formed from quaternary ammonium compounds. In an embodiment, the quaternary ammonium compounds for forming the cationic guar polymer conform to the general formula 1:

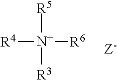

wherein where R³, R⁴ and R⁵ are methyl or ethyl groups; R⁶ is either an epoxyalkyl group of the general formula 2:

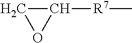

or R⁶ is a halohydrin group of the general formula 3:

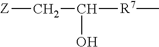

wherein R⁷ is a C₁ to C₃ alkylene; X is chlorine or bromine, and Z is an anion such as Cl—, Br—, I— or HSO₄—.

In an embodiment, the cationic guar polymer conforms to the general formula 4:

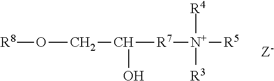

wherein R⁸ is guar gum; and wherein R⁴, R⁵, R⁶ and R⁷ are as defined above; and wherein Z is a halogen. In an embodiment, the cationic guar polymer conforms to Formula 5:

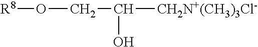

Suitable cationic guar polymers include cationic guar gum derivatives, such as guar hydroxypropyltrimonium chloride. In an embodiment, the cationic guar polymer is a guar hydroxypropyltrimonium chloride. Specific examples of guar hydroxypropyltrimonium chlorides include the Jaguar® series commercially available from Rhone-Poulenc Incorporated, for example Jaguar® C-500, commercially available from Rhodia. Jaguar® C-500 has a charge density of 0.8 meq/g and a M.Wt. of 500,000 g/mole. Jaguar® C-17, which has a cationic charge density of about 0.6 meq/g and a M.Wt. of about 2.2 million g/mol and is available from Rhodia Company. Jaguar® C 13S which has a M.Wt. of 2.2 million g/mol and a cationic charge density of about 0.8 meq/g (available from Rhodia Company). Other suitable guar hydroxypropyltrimonium chloride are: guar hydroxypropyltrimonium chloride which has a charge density of about 1.1 meq/g and a M.Wt. of about 500,000 g/mole is available from ASI, a charge density of about 1.5 meq/g and a M.Wt. of about 500,000 g/mole is available from ASI. Other suitable guar hydroxypropyltrimonium chloride are: Hi-Care 1000, which has a charge density of about 0.7 meq/g and a M.Wt. of about 600,000 g/mole and is available from Rhodia; N-Hance 3269 and N-Hance 3270, which has a charge density of about 0.7 meq/g and a M.Wt. of about 425,000 g/mole and is available from ASI; N-Hance 3196, which has a charge density of about 0.8 and a M. Wt. Of about 1,100,000 g/mole and is available from ASI. AquaCat CG518 has a charge density of about 0.9 meq/g and a M.Wt. of about 50,000 g/mole and is available from ASI. BF-13, which is a borate (boron) free guar of charge density of about 1.1 meq/g and M. W.t of about 800,000 and BF-17, which is a borate (boron) free guar of charge density of about 1.7 meq/g and M. W.t of about 800,000 both available from ASI.

(2) Cationic Non-Guar Galactomannan Polymers

The shampoo compositions of the present invention comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β(1-4) glycosidic linkages. The galactose branching arises by way of an α(1-6) linkage. The ratio of mannose monomers to galactose monomers varies according to the species of the plant and also is affected by climate. Non Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

In one embodiment of the invention, the non-guar galactomannan polymer derivatives have a M. Wt. from about 1,000 to about 10,000,000, and/or form about 5,000 to about 3,000,000.

The shampoo compositions of the present invention include galactomannan polymer derivatives which have a cationic charge density from about 0.5 meq/g to about 7 meq/g. In one embodiment of the present invention, the galactomannan polymer derivatives have a cationic charge density from about 1 meq/g to about 5 meq/g. The degree of substitution of the cationic groups onto the galactomannan structure should be sufficient to provide the requisite cationic charge density.

In one embodiment of the present invention, the galactomannan polymer derivative is a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general formulas 1-5, as defined above.

Cationic non-guar galactomannan polymer derivatives formed from the reagents described above are represented by the general formula 6:

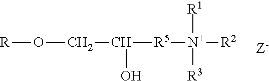

wherein R is the gum. The cationic galactomannan derivative can be a gum hydroxypropyltrimethylammonium chloride, which can be more specifically represented by the general formula 7:

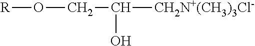

In another embodiment of the invention, the galactomannan polymer derivative is an amphoteric galactomannan polymer derivative having a net positive charge, obtained when the cationic galactomannan polymer derivative further comprises an anionic group.

In one embodiment of the invention the cationic non-guar galactomannan has a ratio of mannose to galactose is greater than about 4:1, a M.Wt. of about 100,000 to about 500,000, and/or from about 150,000 to about 400,000 and a cationic charge density from about 1 meq/g to about 5 meq/g, and/or from 2 meq/g to about 4 meq/g and is a derived from a cassia plant.

The shampoo compositions of the present invention comprise at least about 0.05% of a galactomannan polymer derivative by weight of the composition. In one embodiment of the present invention, the shampoo compositions comprise from about 0.05% to about 2%, by weight of the composition, of a galactomannan polymer derivative.

(3) Cationically Modified Starch Polymer

The shampoo compositions of the present invention comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The shampoo compositions of the present invention comprise cationically modified starch polymers at a range of about 0.01% to about 10%, and/or from about 0.05% to about 5%, by weight of the composition.

The cationically modified starch polymers disclosed herein have a percent of bound nitrogen of from about 0.5% to about 4%.

The cationically modified starch polymers for use in the shampoo compositions of the present invention have a molecular weight from about 850,000 to about 15,000,000 and/or from about 900,000 to about 5,000,000. As used herein, the term “molecular weight” refers to the weight average molecular weight. The weight average molecular weight may be measured by gel permeation chromatography (“GPC”) using a Waters 600E HPLC pump and Waters 717 auto-sampler equipped with a Polymer Laboratories PL Gel MIXED-A GPC column (Part Number 1110-6200, 600.times.7.5 mm, 20 um) at a column temperature of 55.degree. C. and at a flow rate of 1.0 ml/min (mobile phase consisting of Dimethylsulfoxide with 0.1% Lithium Bromide), and using a Wyatt DAWN EOS MALLS (multi-angle laser light scattering detector) and Wyatt Optilab DSP (interferometric refractometer) detectors arranged in series (using a dn/dc of 0.066), all at detector temperatures of 50° C., with a method created by using a Polymer Laboratories narrow dispersed Polysaccharide standard (Mw=47,300), with an injection volume of 200 μl.

The shampoo compositions of the present invention include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers of the present invention generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable .sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof.

In one embodiment of the present invention, cationically modified starch polymers are selected from degraded cationic maize starch, cationic tapioca, cationic potato starch, and mixtures thereof. In another embodiment, cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers in the present invention may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance.gtoreq.80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Suitable cationically modified starch for use in compositions of the present invention is available from known starch suppliers. Also suitable for use in the present invention is nonionic modified starch that could be further derivatized to a cationically modified starch as is known in the art. Other suitable modified starch starting materials may be quaternized, as is known in the art, to produce the cationically modified starch polymer suitable for use in the invention.

Starch Degradation Procedure: In one embodiment of the present invention, a starch slurry is prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

(4) Cationic Copolymer of an Acrylamide Monomer and a Cationic Monomer

According to an embodiment of the present invention, the shampoo composition comprises a cationic copolymer of an acrylamide monomer and a cationic monomer, wherein the copolymer has a charge density of from about 1.0 meq/g to about 3.0 meq/g. In an embodiment, the cationic copolymer is a synthetic cationic copolymer of acrylamide monomers and cationic monomers.

In an embodiment, the cationic copolymer comprises:
(i) an acrylamide monomer of the following Formula AM:

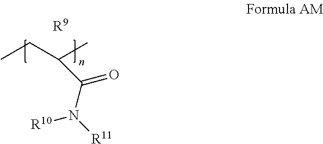

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM:

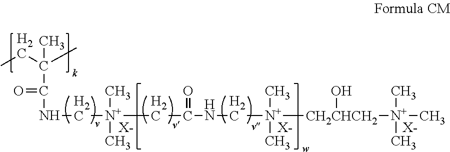

where k=1, each of v, v′, and v″ is independently an integer of from 1 to 6, w is zero or an integer of from 1 to 10, and X⁻ is an anion.

In an embodiment, cationic monomer conforming to Formula CM and where k=1, v=3 and w=0, z=1 and X⁻ is Cl⁻ to form the following structure:

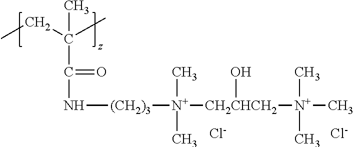

The above structure may be referred to as diquat. In another embodiment, the cationic monomer conforms to Formula CM and wherein v and v″ are each 3, v′=1, w=1, y=1 and X⁻ is Cl⁻, such as:

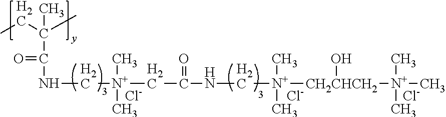

The above structure may be referred to as triquat.

In an embodiment, the acrylamide monomer is either acrylamide or methacrylamide.

In an embodiment, the cationic copolymer (b) is AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride. AM:TRIQUAT is also known as polyquaternium 76 (PQ76). AM:TRIQUAT may have a charge density of 1.6 meq/g and a M.Wt. of 1.1 million g/mol.

In an alternative embodiment, the cationic copolymer is of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

In an embodiment, the cationic copolymer comprises a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

In an embodiment, the cationic copolymer is water-soluble. In an embodiment, the cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. In an embodiment, cationized esters of the (meth)acrylic acid containing a quaternized N atom are quaternized dialkylaminoalkyl (meth)acrylates with C1 to C3 in the alkyl and alkylene groups. In an embodiment, the cationized esters of the (meth)acrylic acid containing a quaternized N atom are selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. In an embodiment, the cationized esters of the (meth)acrylic acid containing a quaternized N atom is dimethylaminoethyl acrylate, which is quaternized with an alkyl halide, or with methyl chloride or benzyl chloride or dimethyl sulfate (ADAME-Quat). In an embodiment, the cationic monomer when based on (meth)acrylamides are quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

In an embodiment, the cationic monomer based on a (meth)acrylamide is a quaternized dialkylaminoalkyl(meth)acrylamide with C1 to C3 in the alkyl and alkylene groups. In an embodiment, the cationic monomer based on a (meth)acrylamide is dimethylaminopropylacrylamide, which is quaternized with an alkyl halide, especially methyl chloride or benzyl chloride or dimethyl sulfate.

In an embodiment, the cationic monomer is a hydrolysis-stable cationic monomer. Hydrolysis-stable cationic monomers can be, in addition to a dialkylaminoalkyl(meth)acrylamide, all monomers that can be regarded as stable to the OECD hydrolysis test. In an embodiment, the cationic monomer is hydrolysis-stable and the hydrolysis-stable cationic monomer is selected from the group consisting of: diallyldimethylammonium chloride and water-soluble, cationic styrene derivatives.

In an embodiment, the cationic copolymer is a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). In an embodiment, the cationic copolymer is formed from acrylamide and acrylamidopropyltrimethylammonium chloride, wherein the acrylamidopropyltrimethylammonium chloride has a charge density of from about 1.0 meq/g to about 3.0 meq/g.

In an embodiment, the cationic copolymer has a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

In an embodiment, the cationic copolymer has a M.Wt. from about 100 thousand g/mol to about 2 million g/mol, or from about 300 thousand g/mol to about 1.8 million g/mol, or from about 500 thousand g/mol to about 1.6 million g/mol, or from about 700 thousand g/mol to about 1.4 million g/mol, or from about 900 thousand g/mol to about 1.2 million g/mol.

In an embodiment, the cationic copolymer is a trimethylammoniopropylmethacrylamide chloride-N-Acrylamide copolymer, which is also known as AM:MAPTAC. AM:MAPTAC may have a charge density of about 1.3 meq/g and a M.Wt. of about 1.1 million g/mol. In an embodiment, the cationic copolymer is AM:ATPAC. AM:ATPAC may have a charge density of about 1.8 meq/g and a M.Wt. of about 1.1 million g/mol.

(5) Cationic Synthetic Polymer

According to an embodiment of the present invention, the shampoo composition comprises a cationic synthetic polymer that may be formed from

i) one or more cationic monomer units, and optionally

ii) one or more monomer units bearing a negative charge, and/or

iii) a nonionic monomer,

wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

In one embodiment, the cationic polymers are water soluble or dispersible, non-crosslinked, and synthetic cationic polymers having the following structure:

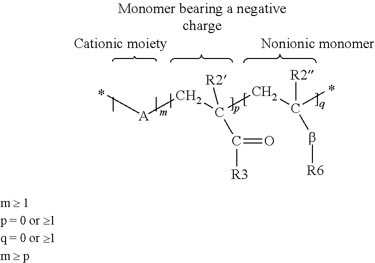

where A, may be one or more of the following cationic moieties:

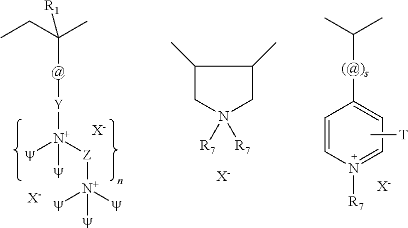

where @=amido, alkylamido, ester, ether, alkyl or alkylaryl;
where Y=C1-C22 alkyl, alkoxy, alkylidene, alkyl or aryloxy;
where ψ=C1-C22 alkyl, alkyloxy, alkyl aryl or alkyl arylox;
where Z=C1-C22 alkyl, alkyloxy, aryl or aryloxy;
where R1=H, C1-C4 linear or branched alkyl;
where s=0 or 1, n=0 or ≧1;
where T and R7=C1-C22 alkyl; and
where X—=halogen, hydroxide, alkoxide, sulfate or alkylsulfate.

Where the monomer bearing a negative charge is defined by R2′=H, C1-C4 linear or branched alkyl and R3 as:

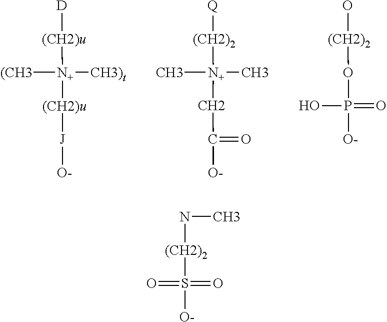

where D=O, N, or S;
where Q=NH₂ or O;
where u=1-6;
where t=0-1; and
where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

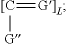

and
where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X−) in association with the synthetic cationic polymers may be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non limiting examples of such counterions include halides (e.g., chlorine, fluorine, bromine, iodine), sulfate and methylsulfate.

In one embodiment, the cationic polymer described herein aids in providing damaged hair, particularly chemically treated hair, with a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer returns the hair to a generally virgin-like, healthier state. Lyotropic liquid crystals are formed by combining the synthetic cationic polymers described herein with the aforementioned anionic detersive surfactant component of the shampoo composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in WO 94/06403 to Reich et al. The synthetic polymers described herein can be formulated in a stable shampoo composition that provides improved conditioning performance, with respect to damaged hair.

Cationic synthetic polymers that can form lyotropic liquid crystals have a cationic charge density of from about 2 meq/gm to about 7 meq/gm, and/or from about 3 meq/gm to about 7 meq/gm, and/or from about 4 meq/gm to about 7 meq/gm. In some embodiments, the cationic charge density is about 6.2 meq/gm. The polymers also have a M. Wt. of from about 1,000 to about 5,000,000, and/or from about 10,000 to about 2,000,000, and/or from about 100,000 to about 2,000,000.

In another embodiment of the invention cationic synthetic polymers that provide enhanced conditioning and deposition of benefit agents but do not necessarily form lytropic liquid crystals have a cationic charge density of from about 0.7 meq/gm to about 7 meq/gm, and/or from about 0.8 meq/gm to about 5 meq/gm, and/or from about 1.0 meq/gm to about 3 meq/gm. The polymers also have a M. Wt. of from about 1,000 to about 5,000,000, from about 10,000 to about 2,000,000, and from about 100,000 to about 2,000,000.

The concentration of the cationic polymers ranges about 0.025% to about 5%, from about 0.1% to about 3%, and/or from about 0.2% to about 1%, by weight of the shampoo composition.

(6) Cationic Cellulose Polymers

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

In an embodiment, the shampoo composition comprises a plurality of cationic conditioning polymers. According to one embodiment, where two cationic conditioning polymers are present, the weight ratio of a first cationic conditioning polymer to a second cationic conditioning polymer is from about 1000:1 to about 2:1. In an embodiment, the weight ratio of the first cationic conditioning polymer to the second cationic conditioning polymer is from about 1000:1 to about 4:1. In an embodiment, weight ratio of the first cationic conditioning polymer to the second cationic conditioning polymer is from about 800:1 to about 4:1, or from about 500:1 to about 4:1, or from about 100:1 to about 5:1, or from about 100:1 to about 6:1, or from about 50:1 to about 6.5:1, or from about 50:1 to about 7:1, or from about 50:1 to about 8.3:1, or from about 50:1 to about 16.7:1

The pH of the composition may be from about pH 3 to about pH 9, or from about pH 4 to about pH 7.

The composition comprises an anti-dandruff active, which may be an anti-dandruff active particulate. In an embodiment, the anti-dandruff active is selected from the group consisting of: pyridinethione salts; azoles, such as ketoconazole, econazole, climbazole and elubiol; selenium sulphide; coal tar, particulate sulfur; keratolytic agents such as salicylic acid; and mixtures thereof. In an embodiment, the anti-dandruff particulate is a pyridinethione salt. Such anti-dandruff particulate should be physically and chemically compatible with the components of the composition, and should not otherwise unduly impair product stability, aesthetics or performance.

Pyridinethione particulates are suitable particulate anti-dandruff actives for use in composition of the present invention. In an embodiment, the anti-dandruff active is a 1-hydroxy-2-pyridinethione salt and is in particulate form. In an embodiment, the concentration of pyridinethione anti-dandruff particulate ranges from about 0.01% to about 5%, by weight of the composition, or from about 0.1% to about 3%, or from about 0.1% to about 2%. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”; zinc pyrithione), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

The anti-dandruff active can also be selected from polyvalent metal salts of pyrithione, one or more anti-fungal and/or anti-microbial actives. Embodiments of the present invention may also comprise a combination of anti-microbial actives.

In an embodiment, the composition comprises an effective amount of a zinc-containing layered material. In an embodiment, the composition comprises from about 0.001% to about 10%, or from about 0.01% to about 7%, or from about 0.1% to about 5% of a zinc-containing layered material, by total weight of the composition.

Many ZLMs occur naturally as minerals. In an embodiment, the ZLM is selected from the group consisting of: hydrozincite (zinc carbonate hydroxide), basic zinc carbonate, aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the composition comprises basic zinc carbonate.

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The composition comprises a cosmetically acceptable carrier. In an embodiment, the carrier is an aqueous carrier. The amount and chemistry of the carrier is selected according to the compatibility with other components and other desired characteristic of the product. In an embodiment, the carrier is selected from the group consisting of: water and water solutions of lower alkyl alcohols. In an embodiment, the carrier is a lower alkyl alcohol, wherein the monohydric alcohol has 1 to 6 carbons. In an embodiment, the carrier is ethanol and/or isopropanol. In an embodiment, the cosmetically acceptable carrier is a cosmetically acceptable aqueous carrier and is present at a level of from about 20% to about 95%, or from about 60% to about 85%.

The composition comprises a surfactant. The surfactant is included to provide cleaning performance to the composition. In an embodiment, the surfactant is selected from the group consisting of: anionic surfactants, amphoteric surfactants, zwitterionic surfactants, cationic surfactants, non-ionic surfactants, and mixtures thereof. In an embodiment, the surfactant is an anionic surfactant. In an embodiment, the composition comprises from about 5% to about 50%, or from about 8% to about 30%, or from about 10% to about 25% of a surfactant, by total weight of the composition.

The composition may comprise a detersive surfactant system. The detersive surfactant system may comprise at least one anionic surfactant, and optionally a co-surfactant selected from the group consisting of: an amphoteric surfactant, a zwitterionic surfactant, a cationic surfactant, a nonionic surfactant, or a mixture thereof. The concentration of the detersive surfactant system in the composition should be sufficient to provide the desired cleaning and lather performance. In an embodiment, the composition comprises from about 5% to about 50%, or from about 8% to about 30%, or from about 10% to about 25% of detersive surfactant system, by total weight of the composition.

In considering the performance characteristics, such as coacervate formation, wet conditioning performance, dry conditioning performance, and conditioning agent deposition on hair, it is desirable to optimize the levels and types of surfactants in order to maximize the performance potential of polymer systems. In one embodiment, the detersive surfactant system for use in the composition comprises an anionic surfactant with an ethoxylate level and an anion level, wherein the ethoxylate level is from about 1 to about 10, and wherein the anion level is from about 1 to about 10. The combination of such an anionic surfactant with the cationic copolymer provides enhanced deposition of conditioning agents to hair and/or skin without reducing cleansing or lathering performance. An optimal ethoxylate level is calculated based on the stoichiometry of the surfactant structure, which in turn is based on a particular M.Wt. of the surfactant where the number of moles of ethoxylation is known. Likewise, given a specific M.Wt. of a surfactant and an anionization reaction completion measurement, the anion level can be calculated.

In an embodiment, the detersive surfactant system comprises at least one anionic surfactant comprising an anion selected from the group consisting of sulfates, sulfonates, sulfosuccinates, isethionates, carboxylates, phosphates, and phosphonates. In an embodiment, the anion is a sulfate.

In an embodiment, the anionic surfactant is an alkyl sulfate or an alkyl ether sulfate. These materials have the respective formulae R⁹OSO₃M and R⁹O(C₂H₄O)ₓSO₃M, wherein R⁹ is alkyl or alkenyl of from about 8 to about 18 carbon atoms, x is an integer having a value of from about 1 to about 10, and M is a cation such as ammonium, an alkanolamine such as triethanolamine, a monovalent metal cation such as sodium and potassium, or a polyvalent metal cation such as magnesium and calcium. Solubility of the surfactant will depend upon the particular anionic surfactants and cations chosen. In an embodiment, R⁹ has from about 8 to about 18 carbon atoms, or from about 10 to about 16 carbon atoms, or from about 12 to about 14 carbon atoms, in both the alkyl sulfates and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. In an embodiment, the alcohols are lauryl alcohol and straight chain alcohols derived from coconut oil or palm kernel oil. Such alcohols are reacted with from about 0 to about 10, or from about 2 to about 5, or about 3, molar proportions of ethylene oxide, and the resulting mixture of molecular species having, for example, an average of 3 moles of ethylene oxide per mole of alcohol is sulfated and neutralized. In an embodiment, the alkyl ether sulphate is selected from the group consisting of: sodium and ammonium salts of coconut alkyl triethylene glycol ether sulfate, tallow alkyl triethylene glycol ether sulfate, tallow alkyl hexa-oxyethylene sulphate, and mixtures thereof. In an embodiment, the alkyl ether sulfate comprises a mixture of individual compounds, wherein the compounds in the mixture have an average alkyl chain length of from about 10 to about 16 carbon atoms and an average degree of ethoxylation of from about 1 to about 4 moles of ethylene oxide. Such a mixture also comprises from about 0% to about 20% C₁₂₋₁₃ compounds; from about 60% to about 100% of C₁₄₋₁₅₋₁₆ compounds; from about 0% to about 20% by weight of C₁₇₋₁₈₋₁₉ compounds; from about 3% to about 30% by weight of compounds having a degree of ethoxylation of 0; from about 45% to about 90% by weight of compounds having a degree of ethoxylation from about 1 to about 4; from about 10% to about 25% by weight of compounds having a degree of ethoxylation from about 4 to about 8; and from about 0.1% to about 15% by weight of compounds having a degree of ethoxylation greater than about 8.

In an embodiment, the anionic surfactant is selected from the group consisting of: ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and mixtures thereof. In addition to the sulfates, isethionates, sulfonates, sulfosuccinates described above, other potential anions for the anionic surfactant include phosphonates, phosphates, and carboxylates.

The composition and/or the detersive surfactant system may comprise a co-surfactant selected from the group consisting of: amphoteric surfactants, zwitterionic surfactants, cationic surfactants, non-ionic surfactants, and mixtures thereof. The concentration of such co-surfactants may be from about 0.5% to about 20%, or from about 1% to about 10%, by total weight of the composition. In an embodiment, the composition comprises a co-surfactant selected from the group consisting of: amphoteric surfactants, zwitterionic surfactants, and mixtures thereof. Non limiting examples of suitable zwitterionic or amphoteric surfactants are described in U.S. Pat. No. 5,104,646 (Bolich Jr. et al.), U.S. Pat. No. 5,106,609 (Bolich Jr. et al.).

Amphoteric surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. In an embodiment, the amphoteric surfactant is selected from the group consisting of: sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof.

In one embodiment, the amphoteric surfactant is a surfactant according to the following structure:

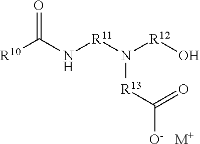

wherein R¹⁰ is a C-linked monovalent substituent selected from the group consisting of: substituted alkyl systems comprising 9 to 15 carbon atoms, unsubstituted alkyl systems comprising 9 to 15 carbon atoms, straight alkyl systems comprising 9 to 15 carbon atoms, branched alkyl systems comprising 9 to 15 carbon atoms, and unsaturated alkyl systems comprising 9 to 15 carbon atoms; and wherein R¹¹, R¹², and R¹³ are each independently selected from the group consisting of: C-linked divalent straight alkyl systems comprising 1 to 3 carbon atoms, and C-linked divalent branched alkyl systems comprising 1 to 3 carbon atoms; and wherein M⁺ is a monovalent counterion selected from the group consisting of sodium, ammonium and protonated triethanolamine. In an embodiment, the amphoteric surfactant is selected from the group consisting of: sodium cocoamphoacetate, sodium cocoamphodiacetate, sodium lauroamphoacetate, sodium lauroamphodiacetate, ammonium lauroamphoacetate, ammonium cocoamphoacetate, triethanolamine lauroamphoacetate, triethanolamine cocoamphoacetate, and mixtures thereof.

In an embodiment, the composition comprises a zwitterionic surfactant, wherein the zwitterionic surfactant is a derivative of an aliphatic quaternary ammonium, phosphonium, and sulfonium compound, in which the aliphatic radicals are straight or branched chain, and wherein one of the aliphatic substituents contains from about 8 to about 18 carbon atoms and one contains an anionic group such as carboxy, sulfonate, sulfate, phosphate or phosphonate. In an embodiment, the zwitterionic surfactant is selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof. In an embodiment, the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

In an embodiment, the co-surfactant is selected from the group consisting of: zwitterionic surfactants, amphoteric surfactants, non-ionic surfactants, and mixtures thereof. In an embodiment, the surfactant is an anionic surfactant and the composition further comprises a co-surfactant, wherein the co-surfactant is selected from the group consisting of: zwitterionic surfactants, amphoteric surfactants, non-ionic surfactants, and mixtures thereof. In an embodiment, the co-surfactant is a non-ionic surfactant selected from the group consisting of: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, and mixtures thereof. In an embodiment, the co-surfactant is a zwitterionic surfactant, wherein the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

Associative Thickeners

Another class of thickeners along with conventional thickeners is associative thickeners. This class contains polymers which modify the rheology of a fluid through associative interactions between polymer chains, the dispersed phase, and the medium. Unlike conventional thickeners, associative thickeners are often times lower molecular weight polymers containing both hydrophilic and hydrophobic regions. The hydrophobic regions are then able to associate with the hydrophobic moieties while the hydrophilic regions are able to associate with the hydrophilic moieties. This can lead to a network formed within a mixture leading to high viscosities and unique rheological properties.

There are various types of associative thickening polymers, such as hydrophobically modified hydroxyethyl celluoloses, hydrophobically modified polypolyacrylates, hydrophobically modified polyacrylic acids, hydrophobically modified polyacrylamides, and hydrophobically modified polyethers.

The class of hydrophobically-modified polyethers include numerous members such as PEG-120-methylglucose dioleate, PEG-N(40 or 60) sorbitan tetraoleate, PEG-150 pentaerythrityl tetrastearate, PEG-55 propylene glycol oleate and PEG-150 distearate. Typically these materials have a hydrophobe, non-limiting examples include cetyl, stearyl, oleayl and combinations thereof, and a hydrophilic portion of repeating ethylene oxide groups with repeat units from 10-300, in an embodiment, from 30-200, and in a further embodiment from 40-150.

The level of associative thickeners, such as PEG-150 distearate, is from about 0.5% to about 3.0%, from about 0.8% to about 2.5%, and from about 1% to about 2%, by weight of the shampoo composition.

Polyols

Polyols are a component of the present invention. In an embodiment of the present invention, a nonlimiting example of a polyol is glycerin. Glycerin is a colorless, odorless, viscous liquid that is very common for use in personal care applications and pharmaceutical formulations. Glycerin contains three hydroxyl groups that are responsible for its solubility in water and its humectant nature. Glycerin is well known as hair and skin benefit agent in personal care applications. This material can penetrate into a human hair to provide conditioning and softness via plasticization of the hair fiber while maintaining a very clean surface feel. Glycerin has been observed to clean more hydrophobic soil components (ie. sebum) than water.

The levels of Glycerin paired with PEG-150 distearate range from about 1.0% to about 10%, from about 2% to about 8% and from about 3.0% to about 6.0% by weight of the shampoo composition.

In another embodiment of the present invention, other polyols may be used. Nonlimiting examples include propylene glycol, sugar polyols such as sorbitol, aloe vera gel and honey.

Silicones

The conditioning agent of the compositions of the present invention can be a silicone conditioning agent. The silicone conditioning agent may comprise volatile silicone, non-volatile silicone, or combinations thereof. The concentration of the silicone conditioning agent typically ranges from about 0.01% to about 10%, by weight of the composition, from about 0.1% to about 8%, from about 0.1% to about 5%, and/or from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which descriptions are incorporated herein by reference. The silicone conditioning agents for use in the compositions of the present invention can have a viscosity, as measured at 25° C., from about 20 to about 2,000,000 centistokes (“csk”), from about 1,000 to about 1,800,000 csk, from about 50,000 to about 1,500,000 csk, and/or from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

Silicone emulsions suitable for use in the embodiments of the present invention include, but are not limited to, emulsions of insoluble polysiloxanes prepared in accordance with the descriptions provided in U.S. Pat. No. 4,476,282 and U.S. Patent Application Publication No. 2007/0276087. Accordingly, suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The average molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscosimeter with spindle 6 at 2.5 rpm. The silicone emulsion may further include an additional emulsifier together with the anionic surfactant,

Other classes of silicones suitable for use in compositions of the present invention include but are not limited to: i) silicone fluids, including but not limited to, silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

In an embodiment, specific classes of silicones suitable for use in compositions of the present invention include but are not limited to:
i) Dimethicone
ii) Dimethiconol
iii) Organo-modified silicones comprising at least one of the following functional groups:
   a) primary, secondary or tertiary amine
   b) quaternary ammonium
   c) alkyl or fluoroalkyl
   d) ether
   e) ester
   f) alcohol
   g) sugar
or combinations of these functional groups; these organomodified silicones can also be random or block copolymers.
iv) Silicone fluids, including but not limited to, silicone oils, which are flowable materials having viscosity less than about 1,000,000 csk as measured at 25° C.;
v) silicone gums; which include materials having viscosity greater or equal to 1,000,000 csk as measured at 25° C.;
vi) silicone resins, which include highly cross-linked polymeric siloxane systems;
vii) high refractive index silicones, having refractive index of at least 1.46, and
viii) mixtures thereof.
   The silicone conditioning material can be a block copolymer made from the following blocks:

a. one polydimethylsiloxane of at least 200 siloxane units

b. polyalkylene oxide linked with amine and quaternary ammonium functional groups optionally, one or more of the blocks can comprise of at least one ester groups. The viscosity of such silicone conditioning material can be 100,000 mPa·s or less.

Organic Conditioning Materials

The conditioning agent of the shampoo compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

Emusifiers

A variety of anionic and nonionic emulsifiers can be used in the shampoo composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

Chelating Agents

The shampoo composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

Levels of the EDDS chelant in the shampoo compositions can be as low as about 0.01 wt % or even as high as about 10 wt %, but above the higher level (i.e., 10 wt %) formulation and/or human safety concerns may arise. In an embodiment, the level of the EDDS chelant may be at least about 0.05 wt %, at least about 0.1 wt %, at least about 0.25 wt %, at least about 0.5 wt %, at least about 1 wt %, or at least about 2 wt % by weight of the shampoo composition. Levels above about 4 wt % can be used but may not result in additional benefit.

Gel Network

The shampoo composition may also comprise fatty alcohol gel networks. These gel networks are formed by combining fatty alcohols and surfactants in the ratio of from about 1:1 to about 40:1, from about 2:1 to about 20:1, and/or from about 3:1 to about 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

The fatty alcohol can be included in the fatty alcohol gel network at a level by weight of from about 0.05 wt % to about 14 wt %. For example, the fatty alcohol may be present in an amount ranging from about 1 wt % to about 10 wt %, and/or from about 6 wt % to about 8 wt %.

The fatty alcohols useful herein include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

Gel network preparation: A vessel is charged with water and the water is heated to about 74° C. Cetyl alcohol, stearyl alcohol, and SLES surfactant are added to the heated water. After incorporation, the resulting mixture is passed through a heat exchanger where the mixture is cooled to about 35° C. Upon cooling, the fatty alcohols and surfactant crystallized to form a crystalline gel network. Table 1 provides the components and their respective amounts for an example gel network composition.

TABLE 1
Gel network components

| Ingredient | Wt. % |
|---|---|
| Water | 78.27% |
| Cetyl Alcohol | 4.18% |
| Stearyl Alcohol | 7.52% |
| Sodium laureth-3 sulfate (28% Active) | 10.00% |
| 5-Chloro-2-methyl-4-isothiazolin-3-one, Kathon CG | 0.03% |

In accordance with embodiments of the present invention, the personal care composition may further comprise one or more benefit agents. Exemplary benefit agents include, but are not limited to, particles, colorants, perfume microcapsules, gel networks, and other insoluble skin or hair conditioning agents such as skin silicones, natural oils such as sun flower oil or castor oil. In an embodiment, the benefit agent is selected from the group consisting of: particles; colorants; perfume microcapsules; gel networks; other insoluble skin or hair conditioning agents such as skin silicones, natural oils such as sun flower oil or castor oil; and mixtures thereof.

The composition forms coacervate particles upon dilution of the composition with water. The percentage of coacervate particles with a floc size of greater than about 20 micron is from about 1% to about 60%. In an embodiment, the percentage of coacervate particles with a floc size of greater than about 20 micron is from about 1% to about 50%, or from about 1% to about 40%, or from about 1% to about 30%, or from about 5% to about 20% from about 5% to about 15%. The floc size is measured after diluting the composition 1:50 dilution with water.

The floc size may be measured using a Lasentec FBRM Method: In a suitable mixing vessel create a 1:9 dilution of composition in distilled water at ambient temperature and mix for 5 min at 250 rpm. Using a peristaltic pump transfer ambient distilled water into the mixing vessel at a rate of 100 g/min resulting in a final dilution of 1:50 parts composition to distilled water. After a 10 min equilibration period a Lasentec Focused Beam Reflectance Method (FBRM) [model S400A available from Mettler Toledo Corp] may be used to determine floc size and amount as measured by chord length and particle counts/sec (counts per sec).

In an embodiment of the method, a mean consumer acceptance rating, on a scale of 20 to 100, of 20 is poor, or 40 is fair, or 60 is good, 80 is very good, and 100 is excellent is achieved. In order to obtain mean consumer acceptance rating values, compositions are evaluated by consumer panels ranging in size from 10 to 400, for example 16 to 310 people. Panelists are asked to use the composition as their only shampoo over a period of time ranging from 3 days to 4 weeks. After use, the panelists are asked to rate different attributes of the composition and its usage experience on a 5 point scale. For the purpose of numerical analysis, the answers are converted to a 100 point scale and the mean consumer acceptance rating calculated.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified.

The following non-limiting Examples are embodiments of the present invention.

### Examples 1-21

### Amphoteric Polymers

Non-limiting examples of the terpolymers are prepared according to the methods found in US 2010/0226868 published on Sep. 9, 2010.

### Key to Abbreviations:

APTAC—acryloylaminopropyl-N,N,N-trimethylammonium chloride
MAPTAC—methyl acryloylaminopropyl-N,N,N-trimethylammonium chloride
AA—acrylic acid
DAA—diallylamine
ADAA EO1—alkoxylated diallylamine with one ethylene oxide
ADAA PO1—alkoxylated diallylamine with one propylene oxide
ADAA EO5—alkoxylated diallylamine with average of five ethylene oxide units
ADAA EO10—alkoxylated diallylamine with average of ten ethylene oxide units

| | | | | | | | | | Molar | |
|---|---|---|---|---|---|---|---|---|---|---|
| | Ter-polymer Composition, Monomer Weight % | | | | | | | | Ratio | |
| Ter- | | | | | ADAA | ADAA | ADAA | ADAA | (M)APTA | |
| polymer | APTAC | MAPTAC | AA | DAA | EO1 | PO1 | EO5 | EO10 | C/AA | MW |
| 1 | 86 | — | 10 | 4 | — | — | — | — | 3.0 | 388000 |
| 2 | 84 | — | 15 | 1 | — | — | — | — | 2.0 | 417000 |
| 3 | 84 | — | 15 | 1 | — | — | — | — | 2.0 | 569000 |
| 4 | 79 | — | 20 | 1 | — | — | — | — | 1.4 | 573000 |
| 5 | 87 | — | 12 | 1 | — | — | — | — | 2.5 | 458000 |
| 6 | 81 | — | 15 | 4 | — | — | — | — | 2.0 | 418000 |
| 7 | — | 80 | 10 | 10 | — | — | — | — | 2.6 | 167000 |
| 8 | 86 | — | 10 | — | 4 | — | — | — | 3.0 | 641000 |
| 9 | 86 | — | 10 | — | 4 | — | — | — | 3.0 | 491000 |
| 10 | 80 | — | 10 | — | 10 | — | — | — | 2.8 | 238000 |
| 11 | 80 | — | 10 | — | — | 10 | — | — | 2.8 | 498000 |
| 12 | — | 80 | 10 | — | 10 | — | — | — | | 141000 |
| 13 | — | 80 | 10 | — | — | 10 | — | — | | 223000 |
| 14 | 80 | — | 10 | — | — | — | 10 | — | 2.8 | 345000 |
| 15 | 86 | — | 10 | — | — | — | 4 | — | 3.0 | 484000 |
| 16 | 84 | — | 15 | — | — | — | 1 | — | 2.0 | 524000 |
| 17 | 86 | — | 10 | — | — | — | — | 4 | 3.0 | 462000 |
| 18 | 84 | — | 15 | — | — | — | — | 1 | 2.0 | 341000 |
| 19 | 83 | — | 14 | 1 | — | — | — | 2 | 2.1 | 468000 |
| 20 | 80 | — | 10 | — | — | — | — | 10 | 2.8 | 315000 |
| 21 | 86 | — | 8 | 4 | — | — | 2 | — | 3.8 | 428000 |

### Example

### Charge Density of Amphoteric Polymers

Charge densities of ter-polymers of the invention are determined at pH4 and pH7 according to the automated titration methods found in Progress in Colloid & Polymer Science, Volume 65, pp. 251-264 (1978). The method is based on the complex formation between cationic and anionic polymers and endpoint determination by cooperative adsorption of a metachromatic dye.

| | Molar Ratio | Charge Density, pH 4 | Charge Density, |
|---|---|---|---|
| Ter-polymer | (APTAC:AA) | (meq/g) | pH 7 (meq/g) |
| 2 | 2.0 | 3.2 | 1.7 |
| 5 | 2.5 | 3.5 | 2.2 |
| 8 | 3.0 | 3.8 | 2.5 |
| 20 | 2.8 | 3.9 | 2.5 |

### Examples 1-15

### Shampoo Compositions (pH 5.5-6.5)

Non-limiting examples of amphoteric ter-polymer containing shampoo compositions 1-15 are prepared according to the methods found herein and in US 2010/0226868 published on Sep. 9, 2010.

| | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| Water | q.s. | q.s. | q.s. | q.s. | q.s. | q.s. | q.s. | q.s. | q.s. | q.s. | q.s. | q.s. | q.s. | q.s. | q.s. | q.s. | q.s. |
| Sodium | 10.5 | 10.5 | 10.5 | 10.5 | 10.5 | 10.5 | 10.5 | 10.5 | 10.5 | 10.5 | 12.0 | 2.0 | 2.0 | 2.0 | 0.5 | 0.5 | 12.0 |
| Laureth | | | | | | | | | | | | | | | | | |
| Sulfate¹ | | | | | | | | | | | | | | | | | |
| Sodium | 1.5 | 1.5 | 1.5 | 1.5 | 1.5 | 1.5 | 1.5 | 1.5 | 1.5 | 1.5 | 1.5 | 1.5 | 1.5 | 1.5 | 1.5 | 1.5 | 1.5 |
| Lauryl Sulfate² | | | | | | | | | | | | | | | | | |
| Cocoamidopropyl Betaine³ | 1.0 | 1.0 | 1.0 | 1.0 | 1.0 | 1.0 | 1.0 | 1.0 | 1.0 | 1.0 | 1.7 | 1.7 | 1.7 | 1.7 | 1.0 | 1.0 | 1.7 |
| Ter- | 0.25 | — | — | — | — | — | — | — | — | — | 0.30 | — | 0.15 | — | — | — | — |
| polymer 1 | | | | | | | | | | | | | | | | | |
| Ter- | — | 0.25 | — | — | — | — | — | — | — | — | — | — | — | — | — | — | — |
| polymer 3 | | | | | | | | | | | | | | | | | |
| Ter- | — | — | 0.25 | — | — | — | — | — | — | — | — | — | — | — | — | — | — |
| polymer 4 | | | | | | | | | | | | | | | | | |
| Ter- | — | — | — | 0.25 | — | — | — | — | — | — | — | — | — | — | — | — | — |
| polymer | | | | | | | | | | | | | | | | | |
| 10 | | | | | | | | | | | | | | | | | |
| Ter- | — | — | — | — | 0.25 | — | — | — | — | — | — | — | — | — | — | — | — |
| polymer | | | | | | | | | | | | | | | | | |
| 11 | | | | | | | | | | | | | | | | | |
| Ter- | — | — | — | — | — | 0.25 | — | — | — | — | — | — | — | — | — | — | — |
| polymer | | | | | | | | | | | | | | | | | |
| 12 | | | | | | | | | | | | | | | | | |
| Ter- | — | — | — | — | — | — | 0.25 | — | — | — | — | — | — | — | — | — | — |
| polymer | | | | | | | | | | | | | | | | | |
| 13 | | | | | | | | | | | | | | | | | |
| Ter- | — | — | — | — | — | — | — | 0.25 | — | — | — | — | — | — | — | — | — |
| polymer | | | | | | | | | | | | | | | | | |
| 15 | | | | | | | | | | | | | | | | | |
| Ter- | — | — | — | — | — | — | — | — | 0.25 | — | — | — | — | — | — | — | — |
| polymer | | | | | | | | | | | | | | | | | |
| 16 | | | | | | | | | | | | | | | | | |
| Ter- | — | — | — | — | — | — | — | — | — | 0.25 | — | 0.30 | — | 0.15 | — | — | — |
| polymer | | | | | | | | | | | | | | | | | |
| 20 | | | | | | | | | | | | | | | | | |
| Polyquaternium-6 | — | — | — | — | — | — | — | — | — | — | — | — | — | — | 0.25 | — | — |
| Cationic | — | — | — | — | — | — | — | — | — | — | — | — | 0.15 | 0.15 | — | 0.25 | 0.30 |
| Guar⁴ | | | | | | | | | | | | | | | | | |
| Dimethiconol⁵ | 1.0 | 1.0 | 1.0 | 1.0 | 1.0 | 1.0 | 1.0 | 1.0 | 1.0 | 1.0 | 0.8 | 0.8 | 0.8 | 0.8 | 0.8 | 1.0 | 0.8 |
| EGDS⁶ | 1.5 | 1.5 | 1.5 | 1.5 | 1.5 | 1.5 | 1.5 | 1.5 | 1.5 | 1.5 | — | — | — | — | 1.5 | 1.5 | — |
| Trihydroxystearin⁷ | — | — | — | — | — | — | — | — | — | — | 0.1 | 0.1 | 0.1 | 0.1 | — | — | 0.1 |
| Fragrance, preservatives, | Up to | Up to | Up to | Up to | Up to | Up to | Up to | Up to | Up to | Up to | Up to | Up to | Up | Up | Up to | Up to | Up to |
| viscosity adjustment | 3% | 3% | 3% | 3% | 3% | 3% | 3% | 3% | 3% | 3% | 3% | 3% | to | to | 3% | 3% | 3% |
| | | | | | | | | | | | | | 3% | 3% | | | |

| | |
|---|---|
| pH adjustment | pH = 5.5-6.5 |

¹Sodium Laureth-1 Sulfate, from Stepan
²Sodium Lauryl Sulfate, from P&G
³Amphosol HCA-B, from Stepan
⁴NHance-3196, from ASI
⁵Small particle dimethiconol, from Wacker
⁶Superol V Glycerine USP, from P&G
⁷EGDS pure, from Evonik
⁸Thixcin R, from Elementis

### Examples 16-25

### Shampoo Compositions (pH 4.0-4.5)

Non-limiting examples of low pH amphoteric ter-polymer containing shampoos are prepared as in example 1-15 at pH 5.5-6.5 with the further addition of an increased level of citric acid and sodium citrate to adjust pH to 4.0-4.5 in full product:

| | 18 | 19 | 20 | 21 | 22 | 23 | 24 | 25 | 26 | 27 | 28 | 29 |
|---|---|---|---|---|---|---|---|---|---|---|---|---|
| Water | q.s. | q.s. | q.s. | q.s. | q.s. | q.s. | q.s. | q.s. | q.s. | q.s. | q.s. | q.s. |
| Sodium Laureth | 10.5 | 10.5 | 13.0 | 13.0 | 13.0 | 13.0 | 13.0 | 13.0 | 13.0 | 13.0 | 10.5 | 13.0 |
| Sulfate¹ | | | | | | | | | | | | |
| Sodium Lauryl | 1.5 | 1.5 | — | — | — | — | — | — | — | — | 1.5 | — |
| Sulfate² | | | | | | | | | | | | |
| Cocoamidopropyl | 1.0 | 1.0 | 1.7 | 1.7 | 1.7 | 1.7 | 1.7 | 1.7 | 1.7 | 1.7 | 1.0 | 1.7 |
| Betaine³ | | | | | | | | | | | | |
| Ter-polymer 1 | 0.25 | — | 0.3 | — | — | — | — | — | 0.15 | — | — | — |
| Ter-polymer 3 | — | 0.25 | — | 0.3 | — | — | — | — | — | — | — | — |
| Ter-polymer 17 | — | — | — | — | 0.3 | — | — | — | — | — | — | — |
| Ter-polymer 18 | — | — | — | — | — | 0.3 | — | — | — | — | — | — |
| Ter-polymer 19 | — | — | — | — | — | — | 0.3 | — | — | — | — | — |
| Ter-polymer 20 | — | — | — | — | — | — | — | 0.3 | — | 0.15 | — | — |
| Cationic Guar⁴ | — | — | — | — | — | — | — | — | 0.15 | 0.15 | 0.25 | 0.3 |
| Dimethiconol⁵ | 0.8 | 0.8 | 0.8 | 0.8 | 0.8 | 0.8 | 0.8 | 0.8 | 0.8 | 0.8 | 0.8 | 0.8 |
| Glycerine⁶ | — | — | — | — | — | — | — | — | — | — | — | — |
| EGDS⁷ | 1.5 | 1.5 | — | — | — | — | — | — | — | — | 1.5 | — |
| Trihydroxystearin⁸ | — | — | 0.1 | 0.1 | 0.1 | 0.1 | 0.1 | 0.1 | 0.1 | 0.1 | — | 0.1 |
| Fragrance, | Up | Up | Up | Up | Up | Up | Up | Up | Up | Up | Up | Up |
| preservatives, | to | to | to | to | to | to | to | to | to | to | to | to |
| viscosity | 3% | 3% | 3% | 3% | 3% | 3% | 3% | 3% | 3% | 3% | 3% | 3% |
| adjustment | | | | | | | | | | | | |

| | |
|---|---|
| Citric | pH = 4.0-4.5 |
| acid/sodium | |
| citrate pH | |
| adjustment | |

¹Sodium Laureth-1 Sulfate, from Stepan
²Sodium Lauryl Sulfate, from P&G
³Amphosol HCA-B, from Stepan
⁴NHance-3196, from ASI
⁵Small particle dimethiconol, from Wacker
⁶Superol V Glycerine USP, from P&G
⁷EGDS pure, from Evonik
⁸Thixcin R, from Elementis

### A. Wet and Dry Conditioning Test Method

This test method is designed to allow for a subjective evaluation of the basic performance of conditioning shampoos for both wet combing and dry combing efficacy. The control treatments exemplified in Table 2 are (1) a clarifying shampoo that employs only surfactants and has no conditioning materials present, and (2) the same clarifying shampoo used in the washing process followed by the application of a mid-range hair conditioner. These treatments facilitate differentiation of performance of a set prototype conditioning shampoos. In a typical test, 3 to 8 separate formulations can be assessed for their performance. The substrate is either virgin brown hair obtainable from a variety of sources that is screened to insure uniformity and lack of meaningful surface damage or low lift bleach damaged hair.

TABLE 2

| Clarifying Shampoo | | Silicone Containing Conditioner | |
|---|---|---|---|
| Formulation (Control 1) | | Formulation (Control 2) | |
| Ingredient | Wt. % | Ingredient | Wt. % |
| Distilled Water | To 100% | Water | To 100% |
| Sodium Laureth-3 Sulfate | 7.00 | L-Glutamic Acid | 0.64 |
| Tetrasodium EDTA | 0.14 | Stearamidoproply- | 2.00 |
| | | dimethylamine | |
| Citric Acid (anhydrous) | 1.11 | Cetyl Alcohol | 2.50 |
| Sodium Citrate (dihydrate) | 0.00 | Stearyl Alcohol | 4.50 |
| Cocamide MEA | 0.50 | Dimethicone/ | 4.20 |
| | | Cyclomethicone | |
| | | (15/85 Blend) | |
| Kathon CG | 0.03 | EDTA | 0.10 |
| Sodium Lauryl Sulfate | 7.00 | Benzyl Alcohol | 0.40 |
| DMDM Hydantoin | 0.10 | Kathon CG | 0.33 |
| Cocoamidopropyl Betaine | 2.00 | Perfume | 0.25 |
| NaCl | 0.70 | dl-Pantyl | 0.225 |
| Perfume | 0.46 | dl-Panthenol | 0.05 |

### B. Treatment Procedure

Five 4 gram, 8 inch length switches are combined in a hair switch holder, wet for ten seconds with manipulation with 40° C. water of medium hardness (9-10 gpg) to ensure complete and even wetting. The switch is deliquored lightly and product is applied uniformly over the length of the combined switches from one inch below the holder towards the tip at a level of 0.1 gram product per one gram of dry hair (0.1 g/g of hair or 2 g for 20 g hair). For more concentrated prototypes the usage level is reduced to 0.05 g/g of hair. The switch combo is lathered for 30 seconds by a rubbing motion typical of that used by consumers and rinsed with 40° C. water flowing at 1.5 gal/min (with the hair being manipulated) for a further 30 seconds to ensure completeness. This step is repeated. For the control treatment with conditioner, it is applied in the same way as shampoo above, manipulated throughout the switch combo and rinsed thoroughly with manipulation, again for 30 seconds. The switches are deliquored lightly, separated from each other, hung on a rack so that they are not in contact and detangled with a wide tooth comb.

### C. Grading Procedures

For wet combing evaluations using trained graders, the switches are separated on the rack into the five sets with one switch from each treatment included in the grading set. Only two combing evaluations are performed on each switch. The graders are asked to compare the treatments by combing with a narrow tooth nylon comb typical of those used by consumers and rate the ease/difficulty on a zero to ten scale. Ten separate evaluations are collected and the results analyzed by a statistical analysis package for establishing statistical significance. Control charting is regularly used to insure that the low and high controls separate into their regular domains. Statistical significance in differences between treatments is determined using Statgraphics Plus 5.1. All conditioning prototypes should be more than two LSDs above the clarifying control to be viewed as acceptable.

For dry combing evaluations, the switches from above are moved into a controlled temperature and humidity room (22° C./50% RH) and allowed to dry overnight. They remain separated as above and panelists are requested to evaluate dry conditioning performance by making three assessments; dry combing ease of the middle of the switch, dry combing ease of the tips, and a tactile assessment of tip feel. The same ten point scale is used for these comparisons. Again, only two panelists make an assessment of each switch set. Statistical analysis to separate differences is done using the same method as above.

### Example

### pH 4.0-4.5 vs. pH 5.5-6.5

Using the wet conditioning method described herein, bleach damaged switches are treated with ter-polymer compositions and graded for ease of wet combing after one cycle (two wash/cycle treatments):

| | | Wet Combing - Body | | | | |
|---|---|---|---|---|---|---|
| Formulation | Polymer | Mean | 95% LSD | | | |
| Low Control 1 | None | 0.5 | | | | D |
| High Control 2 | | 8.6 | A | | | |
| Example 1 | Ter-polymer 1 | 8.1 | A | | | |
| Example 18 | | 8.5 | A | | | |
| Example 2 | Ter-polymer 2 | 6.1 | | B | | |
| Example 19 | | 7.8 | A | | | |
| Comparative Example 16 | Cationic Guar | 4.2 | | | C | |
| Comparative Example 28 | | 5.3 | | B | C | |

As can be seen, ter-polymer compositions of the present invention provide enhanced wet conditioning performance at low pH (4.0-4.5) vs. shampoo compositions at a conventional pH of about 5.5-6.5.

### Example Balanced Deposition of Silicone 1

Using the wet conditioning method described herein virgin brown (VB) switches and bleach damaged (LL) switches are treated with various ter-polymer compositions and extracted for silicone analysis after one cycle (two wash/rinse treatments):

| | | Ratio of Silicone |
|---|---|---|
| | | Deposition |
| Formulation | Polymer | VB:LL |
| Low Control 1 | None | N/A |
| High Control 2 | | 1.21 |
| Example 1 | Ter-polymer 1 | 5.92 |
| Example 2 | Ter-polymer 3 | 1.29 |
| Example 3 | Ter-polymer 4 | 1.50 |
| Example 4 | Ter-polymer 10 | 4.67 |
| Example 8 | Ter-polymer 15 | 4.70 |
| Example 9 | Ter-polymer 16 | 1.89 |
| Comparative Example 15 | Polyquaternium-6 | 5.14 |
| Comparative Example 16 | Cationic Guar | 3.76 |

As demonstrated above, ter-polymer compositions of the present invention provide more balanced deposition of silicone benefits agents across hair substrates.

### Example Balanced Deposition of Silicone

Using the wet conditioning method described herein virgin brown (VB) switches and bleach damaged (LL) switches are treated with ter-polymer compositions at pH 4.0-4.5, a total of five cycles (total of ten shampoo wash/rinse treatments), graded for ease of wet and dry combing and extracted for silicone analysis:

| | | Wet Combing - | | | | Dry Combing - | | | | | Ratio of Si |
|---|---|---|---|---|---|---|---|---|---|---|---|
| | | Body (LL) | | | | Body (LL) | | | | | Deposition |
| Formulation | Polymer | Mean | 95% LSD | | | Mean | 95% LSD | | | | VB:LL |
| Low Control 1 | None | 0.3 | | | C | 1.6 | | | | D | |
| High Control 2 | | 7.0 | A | | | 8.4 | | B | | | 2.10 |
| Example 20 | Ter-polymer 1 | 7.3 | A | | | 9.4 | A | | | | 2.63 |
| Example 21 | Ter-polymer 3 | 8.0 | A | | | 9.1 | A | B | | | 1.20 |
| Example 22 | Ter-polymer 17 | 7.6 | A | | | 9.3 | A | B | | | 1.99 |
| Example 23 | Ter-polymer 18 | 6.8 | A | | | 9.3 | A | B | | | 1.89 |
| Example 24 | Ter-polymer 19 | 7.5 | A | | | 9.3 | A | B | | | 1.37 |
| Example 25 | Ter-polymer 20 | 8.1 | A | | | 9.3 | A | B | | | 2.15 |
| Comparative | Cationic Guar | 3.0 | | B | | 7.4 | | | C | | 2.18 |
| Example 29 | | | | | | | | | | | |

As demonstrated above, low pH ter-polymer compositions of the present invention provide strong wet and dry conditioning benefits on bleach damaged hair and more balanced deposition of silicone benefits agents across hair substrates.

### Example

### APTAC vs. MAPTAC

Using the wet conditioning method described herein bleach damaged switches are treated with ter-polymer compositions and graded for ease of wet combing after one cycle

| | | | | | | | | Dry Combing - | | | | | |
|---|---|---|---|---|---|---|---|---|---|---|---|---|---|
| | | Wet Combing - Body | | | | | | Body | | | | | Silicone |
| Formulation | Polymer | Mean | 95% LSD | | | | | Mean | 95% LSD | | | | (ppm) |
| Low Control 1 | None | 0.4 | | | | | E | 1.5 | | | | D | 4 |
| High Control 2 | | 9.4 | A | | | | | 9.0 | A | B | | | 60 |
| Example 4 | Ter-polymer 10 | 6.3 | | | C | | | 8.5 | A | B | | | 107 |
| Example 5 | Ter-polymer 11 | 8.4 | A | B | | | | 9.4 | A | | | | 195 |
| Example 6 | Ter-polymer 12 | 3.1 | | | | D | | 6.9 | | | C | | 55 |
| Example 7 | Ter-polymer 13 | 4.3 | | | | D | | 6.8 | | | C | | 62 |

As demonstrated above, APTAC based ter-polymers 10/11 provide stronger benefits than the equivalent MAPTAC based ter-polymers 12/13 as measured by 1) intrinsic wet and dry conditioning, and 2) absolute silicone deposition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A personal care composition comprising:
a) from about 5% to about 50% of a surfactant;
b) from about 0.01% to about 10% of a conditioning agent;
c) from about 0.05 to about 5 weight percent of the personal care composition of an amphoteric ter-polymer of the following:
i.) a cationic monomer encompassed by formula (I), 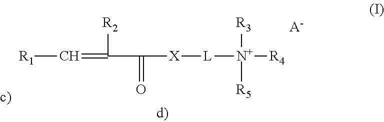
in which:
R₁ and R₂ are independently hydrogen or methyl, R₃, R₄ and R₅ are independently linear or branched C₁-C₄ alkyl radicals, X is NH, NR₆ or oxygen, wherein R₆ is C₁-C₄ alkyl, L is CₙH₂ₙ, n is an integer from 1 to 5, and A⁻ is an anion derived from an organic or inorganic acid, such as a methosulphate anion or halide, such as chloride or bromide,
ii.) at least one anionic monomer selected from the group consisting of ethylenically unsaturated carboxylic acid and sulfonic acid containing monomers; and
iii.) a diallylamine monomer defined by formula (II), 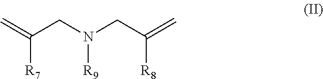
in which:
R₇ and R₈ are independently hydrogen or C₁-C₄ alkyl, and R₉ is hydrogen, branched or linear C₁-C₃₀ alkyl, *[AO]ₘ—R₁₀, C₁-C₃₀ alkoxy, hydroxy substituted alkyl, alkylphenyl, carboxyalkyl, alkoxyalkyl and carboxyamidalkyl; AO is a C₁-C₄ alkylene oxide or mixtures of two or more types thereof, it being possible for the two or more types to be attached to one another in block form or in random form, m is an integer from 2 to 200, R₁₀ is hydrogen or methyl; and
wherein the ratio of cationic monomer i) to anionic monomer ii) is from about 5 to about 1.

2. A personal care composition according to claim 1 comprising wherein the ratio of cationic monomer i) to anionic monomer ii) is from about 3 to about 1.

3. A personal care composition according to claim 1 wherein the amphoteric ter-polymer has a molecular weight of about 100,000 to about 1,500,000.

4. A personal care composition according to claim 3 wherein the amphoteric ter-polymer has a molecular weight of about 200,000 to about 1,000,000.

5. A personal care composition according to claim 1 wherein the amphoteric ter-polymers are composed of at least three monomers:
i.) a cationic monomer of the formula: 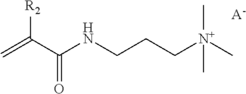
in which R₂ is hydrogen or methyl and A is an anion derived from organic or inorganic acid.
ii.) anionic monomers from the group of ethylenically unsaturated carboxylic acids of formula: 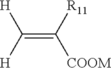
where R₁₁ is hydrogen or methyl and M is hydrogen, a monovalent metal ion, ammonium or organic ammonium ion.
iii.) one or more non-ionic monomers selected from diallylamine (DAA) or diallylamine derivatives of the formula: 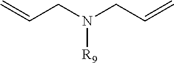
where R₉ is —[AO]ₘ—R₁₀, AO is ethyleneoxide or propyleneoxide or mixtures thereof, m=1-100 and R₁₀ is hydrogen or methyl.

6. A personal care composition according to claim 1 wherein the cationic monomer is acrylamidopropyltrimethyl ammonium chloride.

7. A personal care composition according to claim 1 wherein the anionic monomer is selected from the group consisting of acrylic acid (AA), 2-acrylamido-2-methylpropane sulfonic acid (AMPSA) and mixtures thereof.

8. A personal care composition according to claim 1 wherein when the pH of the composition is in the range of about 3.5 to about 4.5.

9. A personal care composition of claim 1, wherein the personal care composition further comprises at least one deposition polymer.

10. A personal care composition of claim 9 wherein the deposition polymer is a cationic polymer.

11. The personal care composition of claim 1, wherein the personal care composition further comprises one or more additional conditioning agents.

12. The personal care composition of claim 11, wherein said one or more additional conditioning agents is a silicone.

13. The personal care composition of claim 1, wherein said hair care composition further comprises one or more additional benefit agents.

14. The personal care composition of claim 13, wherein said one or more additional benefit agents is selected from the group consisting of anti-dandruff agents, vitamins, chelants, perfumes, brighteners, enzymes, sensates, attractants, anti-bacterial agents, dyes, pigments, bleaches, and mixtures thereof.

15. The personal care composition of claim 1, further comprising a dispersed gel network phase comprising:
i. at least about 0.05% of one or more fatty alcohols, by weight of said hair care composition;
ii. at least about 0.01% of one or more gel network surfactants, by weight of said hair care composition; and
iii. water.
